(19) United States
(12) Patent Application Publication
(10) Pub. No.: US 2014/0276672 A1
(43) Pub. Date: Sep. 18, 2014

(54) STABLE MODE-LOCKED LASER APPARATUS

(71) Applicant: AMO Development LLC., Santa Ana, CA (US)

(72) Inventors: John WY Lee, San Jose, CA (US); Jian He, San Jose, CA (US); Jiandong Xu, San Jose, CA (US); Donald Simpson, Orange, CA (US); Gennady Imeshev, Irvine, CA (US); Oleg J. Korovyanko, Lake Forest, CA (US); Zenon Witowski, Rancho Santa Margarita, CA (US)

(21) Appl. No.: 14/198,319

(22) Filed: Mar. 5, 2014

Related U.S. Application Data
(60) Provisional application No. 61/794,044, filed on Mar. 15, 2013.
(60) Provisional application No. 61/792,799, filed on Mar. 15, 2013.

(51) Int. Cl. A61F 9/008

(52) U.S. Cl. CPC A61F 9/00814
USPC 606/4

(57) ABSTRACT

Embodiments of this invention are directed to a laser system configured to deliver a pulsed laser beam to a patient's eye. The system includes a laser engine comprising an optically-pumped laser oscillator configured with an extracavity waveplate, and an optional intracavity waveplate, that can be tilted and rotated to provide a limited range of wavelengths for laser mode excitation and to maintain stable mode-locked laser operation. In an embodiment, the present design includes an oscillator and a photosensor, such as a fast photodetector or an autocorrelator, positioned to receive a beam of laser light associated with the oscillator or laser engine, and a controller configured to receive readings from the photosensor and alter the laser gain provided within the oscillator to a level outside the bistable performance zone avoiding mode and gain competitions.

### CROSS-REFERENCE TO RELATED APPLICATIONS

This application claims priority to U.S. Provisional Application No. 61/794,044 filed Mar. 15, 2013 and U.S. Provisional Application No. 61/792,799 filed Mar. 15, 2013, which are hereby incorporated by reference in their entirety.

### BACKGROUND

1. Technical Field

Embodiments of the present invention generally relate to laser engines, and more specifically to the application of laser pulses during surgical procedures such as ocular surgical procedures.

2. Background

Eye surgery is now commonplace with some patients pursuing it as an elective procedure to avoid using contact lenses or glasses and others pursuing it to correct adverse conditions such as cataracts. Moreover, with recent developments in laser technology, laser surgery has become the technique of choice for ophthalmic procedures. Laser eye surgery typically uses different types of laser beams, such as ultraviolet lasers, infrared lasers, and near-infrared, ultra-short pulsed lasers, for various procedures and indications.

A surgical laser beam is preferred over manual tools like microkeratomes as it can be focused accurately on extremely small amounts of ocular tissue, thereby enhancing precision and reliability. For example, in the commonly-known LASIK (Laser Assisted In Situ Keratomileusis) procedure, an ultra-short pulsed laser is used to cut a corneal flap to expose the corneal stroma for photoablation with an excimer laser. Ultra-short pulsed lasers emit radiation with pulse durations as short as 10 femtoseconds and as long as 3 nanoseconds, and a wavelength between 300 nm and 3000 nm. Besides cutting corneal flaps, ultra-short pulsed lasers are used to perform cataract-related surgical procedures, including capsulorhexis, capsulotomy, as well as softening and/or breaking of the cataractous lens.

The laser engine employed in non-UV, ultra-short pulse laser surgery delivers a beam of laser pulses to the eye of the patient. Components of the laser engine typically include a mode-locked oscillator, configured to deliver pulses having femtosecond or picosecond durations. Such a mode-locked oscillator typically produces one pulse per round trip through the oscillator cavity. Under certain conditions, such an oscillator could produce more than one pulse per round trip through the cavity. The pulses are then amplified or partially amplified in an amplifier that is generally part of the laser engine. The pulses are then delivered to the output of the ophthalmic surgical system. More than one pulse, such as two pulses per round trip, could lead to less than ideal tissue ablation and photodisruption quality. Double pulses could also lead to an increased ablation energy observable using a model material such as an Agarose gel.

Typically, it is difficult and time consuming to identify and ensure operating parameters where an oscillator could only produce a single pass per round trip rather than two or more pulses per round trip. Solving the issue would normally require one or more specialized tools and techniques. Moreover, such a single-pulse parameter range is affected by small variations in the properties of one or more components in or properties of an oscillator, such as gain of active medium, pump diode performance, SESAM parameters, mirrors' reflectivities, losses, etc. For these reasons the single-pass operating regime could vary and would be difficult to precisely predict a priori.

In addition, such lasers are typically of single frequency and operation in most instances calls for selecting a mode and subsequently maintaining the mode and pulse profile for an extended period of time. Switching between modes can result in certain instabilities, particularly over time, affecting the duration and peak power of ultra-short laser pulses. The result may be variability in the ablation or photodisruption quality of a laser refractive surgical system. In many cases, pulse width and peak power issues resulting from mode switching in a mode locked laser are consequences of mode competition and gain competition in a laser resonator such as a laser oscillator.

One phenomenon resulting from mode locked oscillator instability is a high transition point resulting from a mode change when excitation power increases, and a low transition from the reverse, i.e. a mode change back to the original mode as excitation power decreases. This phenomenon is known as bi-stability. The device tends to switch between two modes at a first high switch point when transitioning from, for example, a lower gain value to a higher gain value, causing a discrete jump in a measured quantity such as pulse duration, but switching at a second low switch point when transitioning from the higher gain value to the lower gain value. In one instance, the switching occurs when mode switches from one pulse per oscillator cavity round trip to two pulses per oscillator cavity round trip, and at a different switch level when transitioning from two pulses to one pulse. A change in mode switching operation may cause various performance issues in the laser engine, such as increased gel cut energy. These nonlinear anomalies may adversely affect the laser's performance, resulting in a loss of mode locking, and in turn, less than ideal surgical outcomes when the laser is used for ophthalmic procedures.

The difficulty with laser bi-stability issues is recognizing when these instabilities occur and compensating for them. As noted, a laser pulse may travel once through the oscillator cavity, but when multiple modes are competing and multiple pulses are intervening, the result may be unstable laser system operation for a period of time, providing imprecise laser cutting pulses, and in a worst case scenario, the patient may be harmed by the laser surgical procedure.

It would be highly beneficial if such mode transition or bi-stability issues could be eliminated, either by filtering out undesirable oscillating modes and restricting to single mode operation, or otherwise reducing the gain in the laser cavity through identification of the mode transition region and operation below this region to maintain laser stability. It would also be highly beneficial if a single-pulse operating regime could be quickly and reliably identified for each oscillator, and if the oscillator could be set to operate in a single-pulse operating regime.

### SUMMARY

Accordingly, an embodiment of the present invention provide a laser engine configured to deliver a pulsed laser beam to a patient's eye, where the includes an optically-pumped laser oscillator configured with a waveplate in front of the pump laser diode and external to the laser resonant cavity, and an optional waveplate within the laser resonant cavity, wherein the waveplate(s) can be tilted and rotated to provide a limited range of wavelengths for laser mode excitation and to maintain stable mode-locked laser operation. Alternately, an embodiment of this invention includes an oscillator and a photosensor, such as a fast photodetector or an autocorrelator, positioned to receive a beam of laser light associated with the oscillator or laser engine, and a controller configured to receive readings from the photosensor and alter laser gain provided within the oscillator to a level outside the bistable performance zone avoiding mode and gain competitions.

This summary and the following detailed description are merely exemplary, illustrative, and explanatory, and are not intended to limit, but to provide further explanation of the invention as claimed. Additional features and advantages of the invention will be set forth in the descriptions that follow, and in part will be apparent from the description, or may be learned by practice of the invention. The objectives and other advantages of the invention will be realized and attained by the structure particularly pointed out in the written description, claims and the appended drawings.

### BRIEF DESCRIPTION OF THE DRAWINGS

Understanding this invention will be facilitated by the following detailed description of the preferred embodiments considered in conjunction with the accompanying drawings, in which like numerals refer to like parts. Note, however, that the drawings are not drawn to scale.

FIG. 1 illustrates a general overview of a non-UV, ultra-short pulse laser arrangement according to an embodiment of this invention.

FIG. 2 is a general diagram of the components of a non-UV, ultra-short pulse laser engine in an ophthalmic surgical laser system.

FIG. 3 illustrates an oscillator that may be employed with the present design

FIG. 4 is a pulse stretcher/compressor according to an embodiment of this invention.

FIG. 5 shows an amplifier that may be employed in an embodiment of this invention.

FIG. 6 illustrates the bi-stability issue with a mode locked laser.

FIG. 7 shows the diode-pumping scheme of an oscillator employing a waveplate according to one embodiment of the present design.

FIG. 8 illustrates an oscillator employing a photosensor, such as a fast photodetector or an autocorrelator, configured to sense laser attributes such that gain of the pump diode can be adjusted to avoid unstable laser operation.

### DETAILED DESCRIPTION

The drawings and related descriptions of the embodiments have been simplified to illustrate elements that are relevant for a clear understanding of these embodiments, while eliminating various other elements found in conventional collagen shields, ophthalmic patient interfaces, and in laser eye surgical systems. Those of ordinary skill in the art may thus recognize that other elements and/or steps are desirable and/or required in implementing the embodiments that are claimed and described. But, because those other elements and steps are well known in the art, and because they do not necessarily facilitate a better understanding of the embodiments, they are not discussed. This disclosure is directed to all applicable variations, modifications, changes, and implementations known to those skilled in the art. As such, the following detailed descriptions are merely illustrative and exemplary in nature and are not intended to limit the embodiments of the subject matter or the uses of such embodiments. As used in this application, the terms “exemplary” and “illustrative” mean “serving as an example, instance, or illustration.” Any implementation described as exemplary or illustrative is not meant to be construed as preferred or advantageous over other implementations. Further, there is no intention to be bound by any expressed or implied theory presented in the preceding background of the invention, brief summary, or the following detailed description.

FIG. 1 illustrates a general overview of a laser arrangement configured to employ the present design. From FIG. 1, the laser engine 100 includes a laser source 101 and provides laser light to a variable attenuator 102 configured to attenuate the beam, then to energy monitors 103 to monitor the beam energy level, and first safety shutter 104 serving as a shutoff device if the beam is unacceptable. The beam steering mirror 105 redirects the resultant laser beam to the beam delivery device 110, through the articulated arm 106 to a range finding camera 111. The range finding camera 111 determines the range needed for the desired focus at the eye 120. The beam delivery device 110 includes second safety shutter 112, a beam monitor 113, a beam pre-expander 114, an X-Y (position) scanner 115, and a zoom beam expander 116. The zoom beam expander 116 expands the beam toward an IR (infrared) mirror 117, which reflects and transmits the received beam. The mirror 118 reflects the received beam to a video camera 119, which records the surgical procedure on the eye 120. The IR mirror 117 also reflects the laser light energy to an objective lens 121, which focuses laser light energy to the eye 120.

In ophthalmic surgery using a pulsed laser beam, non-UV, ultra-short pulse laser technology can emit pulsed radiation having ultra-short pulse durations measured in as long as a few nanoseconds or as short as a few femtoseconds. Such a device as shown in FIG. 1 can provide an intrastromal photodisruption technique for reshaping the cornea using a non-UV, ultra-short (e.g., of femtosecond pulse duration), pulsed laser beam produced by the laser source 101 that propagates through corneal tissues and focuses at a point below the surface of the cornea to photodisrupt stromal tissues.

Although the system may be used to photoalter a variety of materials (e.g., organic, inorganic, or a combination thereof), the system is suitable for ophthalmic applications in one embodiment. The focusing optics, such as the beam pre-expander 114, the zoom beam expander 116, the IR mirror 117 and the objective lens 121, direct the pulsed laser beam toward the eye 120 (e.g., onto or into the cornea) for plasma mediated (e.g., non-UV) photoablation of superficial tissues, or into the stroma of the cornea for intrastromal photodisruption of tissues. In this embodiment, the system may also include a lens to change the shape (e.g., flatten or curve) of the cornea prior to scanning the pulsed laser beam toward the eye. The system is capable of generating the pulsed laser beam with physical characteristics similar to those of the laser beams generated by a laser system disclosed in U.S. Pat. No. 4,764,930, and U.S. Pat. No. 5,993,438, which are incorporated here by reference.

The ophthalmic laser system can produce an ultra-short pulse laser beam for use as an incising laser beam. This pulsed laser beam preferably has laser pulses with durations as long as a few nanoseconds or as short as a few femtoseconds. For intrastromal photodisruption of tissues, the pulsed laser beam works at a wavelength that permits it to pass through the cornea without absorption by the corneal tissues. The wavelength of the pulsed laser beam is generally in the range of about 300 nm to about 3000 nm, and the irradiance of the pulsed laser beam for accomplishing photodisruption of stromal tissues at the focal point is typically greater than the threshold for optical breakdown of the tissues. Although a non-UV, ultra-short pulse laser beam is described in this embodiment, the pulsed laser beam may have different pulse durations and wavelengths in other embodiments. Further examples of devices employed in performing ophthalmic laser surgery are disclosed in, for example, U.S. Pat. Nos. 5,549,632, 5,984,916, and 6,325,792, which are incorporated here by reference.

FIG. 2 illustrates a general overview of the components of the laser engine 101. From FIG. 2, there is provided an oscillator 201, a pulse stretcher/compressor 202, and an amplifier 203. A controller 204 may be provided in the embodiments discussed herein. Lasers producing pulses in the femtosecond duration range operate and generate pulses at high peak power levels, and if left unaltered can damage laser intracavity components. To address this issue, chirped pulse amplification (CPA) is employed wherein the pulse duration is extended or stretched to the picosecond range, resulting in a significant reduction in the pulse peak power. From FIG. 2, the oscillator 201 generates and outputs a beam of femtosecond laser pulses. The pulse stretcher/compressor 202 extends the duration of the received pulses from the oscillator. The amplifier 203 samples these pulses and increases the individual pulse energy by multi-passing over the amplifier gain medium. The pulse stretcher/compressor then recompresses the amplified pulses to the femtosecond range prior to delivery.

FIG. 3 illustrates an oscillator 301 used in a femtosecond laser surgical device. The oscillator 301 includes a laser pump 302, which directs laser excitation energy through a focusing lens 303A and a dichroic element 303B to the oscillator glass assembly 304, horizontally polarized at Brewster's angle. The oscillator laser cavity consists of an output coupler 311, reflective surfaces 303B, 305, 306, 307 and 309, a gain medium 304, and a mode-locking element 308 functioning as an end-cavity reflective surface. Ultra-short pulse laser light energy ultimately passes out of the oscillator 301, to a reflective surface 312, through a beam splitter 313, and into a pulse stretcher/compressor 202, not shown in this view.

FIG. 4 illustrates the components of a pulse stretcher/compressor 401, which receives the pulsed oscillator beam under a half mirror 402, through a half wave plate 403, and onto a reflective surface 404, a half mirror 405, a grating 406, a stretcher lens 407, a folding mirror 408, and a stretcher mirror 409. The beam is reflected and travels back through the elements 408, 407 and 406 to the half mirror 405. It bounces back and forth a number of times over the path sequence through the grating 406 and the above optical elements before emerging out of the half mirror 405 and onto the elements 404 and 403. The beam is then reflected by the half mirror 402 as well as the reflective surface 410, and continues its path onto a three-port Faraday isolator 411 configured to provide polarization rotation of forward and reverse propagating beams to reflective surfaces 412 and 420, respectively. As shown, the beam of stretched oscillator pulses passes through a reflective surface 412, a half wave plate 413, and another reflective surface 414, and provides pulse seeding to the amplifier (not shown in this view). The beam of amplified pulses emerging from the amplifier travels through the elements 414, 413, and 412, enters the reverse side of the Faraday isolator 411, and arrives at the half mirror 420. Compression of amplifier pulses proceeds from beam propagation through the half mirror 420, to the reflective surface 419, through the grating 406, to the compressor retro-reflection assembly 415 comprising the reflective surfaces 416 and 417, back through the grating 406, and to the reflective surface 418. These pulses are reflected and further compressed by passing through the grating 406, the retro-reflection assembly 415, the grating 406 again, the reflective surface 419, and the half mirror 420. The beam of compressed pulses travels onto the reflective surface 421, the folding mirror 422, the energy wheel 423, the beam splitters 424 and 425, and the fast shutter 426, before arriving at the articulating arm 427. Light from beam splitters 424 and 425 are directed to the other components of the surgical system.

FIG. 5 illustrates one embodiment of an amplifier 501, again including a number of reflective surfaces 502, 505, 507, 510, 511, and 512, as well as a photodiode for pulse detection 503, a polarizer assembly 504, a Pockels cell 506, and a Q-switch photo diode 508. The reflective surface 512 operates on a translation device. Other components of the amplifier are the amplifier glass assembly 513, the focusing lenses 514, and the pump laser diode 515.

### Waveplate

FIG. 6 illustrates a typical problem encountered in a system such as that shown in FIGS. 3-5. From FIG. 6, the output pulse-width of the oscillator decreases with increasing pump laser diode current until the current hits approximately 852 mA, where the output pulse-width jumps abruptly from 132 femtoseconds to 223 femtoseconds, and again decreases with increasing current. When current is decreased, the current passes through 850 mA without mode transition and output pulse-width increases to approximately 261 femtoseconds before switching at 704 mA. During switching, the oscillator output pulse-width falls to 170 femtoseconds. The lower branch at currents below 852 mA corresponds to a single pulse in the cavity, while the upper branch at currents above 704 mA corresponds to two pulses in the cavity. For currents below 704 mA only one pulse exists in the cavity. Such a regime is termed as a “single-pulse” regime. For currents above 704 mA and below 852 mA either one or two pulses are possible in the cavity. Such a regime of operation is termed a “bistable” regime. For currents above 852 mA two pulses exist in the cavity. Such a regime is termed as a “double-pulse” regime. For even higher currents more than two pulses could exist in the cavity at a given time.

The present design includes a waveplate to alter performance such as is shown in FIG. 6, and, as an alternative arrangement, monitoring of operation and setting of operating gain or current at levels that do not provide the hysteresis shown in FIG. 6.

A birefringent etalon, in the form of a crystal quartz retardation waveplate, is employed to alter the spectral and polarization properties of the multimode laser diode pumping the oscillator laser glass at Brewster's angle. From FIG. 3, the birefringent etalon may be provided between pump laser diode 302 and oscillator glass assembly 304. The birefringent etalon enables selection of a preferential, dominating laser mode and discrimination or decoupling of a competing mode in the resonant cavity.

The free spectral range (FSR) of an etalon defining the wavelength separation between adjacent transmission peaks is given by:

$$Δ λ = \frac{{λ}_{0}^{2}}{2 nl cos θ + {λ}_{0}} \approx \frac{{λ}_{0}^{2}}{2 nl cos θ} ,$$

where λ₀ is the central wavelength of the nearest transmission peak, n is the index of refraction, l the thickness of the etalon, and θ the angle of refraction of the input beam.

The exact FSR requirement for the etalon in the laser depends on the amount of inhomogeneous to homogeneous broadening in the gain medium and on any spatial hole-burning effects, as well as on the multimode emission of the pump laser diode. A retardation plate is adopted for polarization selection of the oscillating mode in the laser. The diode light is not linearly-polarized, as the individual mode component may not have the same polarization. The retardation plate, together with the laser glass providing linear polarization at the Brewster's angle, can therefore select the desirable diode mode for optical pumping and suppress the other unwanted diode laser modes.

A half-wave, a quarter-wave, or an eighth-waveplate can be used as the retardation plate. The quarter-wave retardation plate generally provides acceptable reflection loss at a reduced angle of incidence for inhibiting mode instability. With the quarter waveplate, the pulse-width monotonically decreases with increasing diode current and increases with decreasing current.

FIG. 7 illustrates the primary components of the present design, including pump diode 701, quarter waveplate 702, focusing lens 703, and oscillator glass assembly at Brewster's angle 704. These components may be employed in an oscillator similar to that shown in FIG. 3, and other components may be included between or outside of the components shown. In one embodiment, the quarter waveplate 702 may be tilted and rotated manually or using computer control such that the bifurcation region of the oscillator is shifted continuously out of the diode current range that is known to produce hi-stability issues.

In one embodiment, a multiple-order waveplate is employed that provides a beneficial variation of retardation with wavelength. The modification of object distance due to tilt and rotation of the waveplate 702 inserted between the pump diode 701 and the focusing lens 703 is minor compared to the variation in beam divergence of the pump diode 701. The waveplate 702 may be anti-reflection-coated on both surfaces. The pump diode 701 operates at a higher current than normal because of the reduction in spectrally-filtered pumping power.

In another embodiment, a second multiple-order waveplate is inserted within the laser resonant cavity for maintaining single mode operation of the oscillator. The waveplate may be tilted and rotated to adjust the lasing wavelength bandwidth such that the number of competing resonant modes excited in the oscillator is limited.

### Measurement and Compensation

An alternative design seeks to measure performance and determine the bistable region, and to provide pump laser diode current levels outside the oscillator mode bi-stability region in the single-pulse region. The present embodiment employs a fast photodetector or an autocorrelator to detect transition points between single and two pulse regimes and sets the oscillator to operate at a current in the desired range, such as at a current level clearly in the single pulse range.

This aspect of the design relates to the operation of a passively mode-locked femtosecond laser oscillator. A laser diode controller with built-in diode current and temperature displays may be employed to operate the pump laser diode and collect measurements. An internal diode driver board typically powers the pump laser diode and an external electronic multimeter may record the measured voltage for the operating diode current, which in one embodiment may be adjusted at a diode current trimming pot on an internal diode driver board. An external fast photodiode detector placed at the oscillator output window monitors the mode-locked laser pulses and these may be displayed or the results collected to analyze the nonlinear properties. The autocorrelator is positioned to measure oscillator pulse width or more generally to generate and measure a signal proportional to the time-averaged square of the peak power.

FIG. 8 illustrates the present operation. The fast photodetector or autocorrelator 802 is placed as shown outside the oscillator 801, after the reflective surface 803, and in front of the beam splitter 804 for detection of pulse signals. The signal received at the autocorrelator 802 is proportional to the time-averaged square of the peak power of the optical pulses. When the oscillator gain is increased starting from the single-pulse regime, the pulse length and average power are approximately the same before and after the switch between single pulse and double pulse, but the autocorrelator signal drops substantially in a short time period as the energy is distributed between two pulses. When the oscillator gain is lowered beginning from the double pulse regime, at some point the oscillator switches from double pulse to single pulse. This autocorrelator detects this transition, where the signal abruptly increases substantially.

Not shown in the view of FIG. 8 is a computing device set to receive signals from autocorrelator 802 and provide commands, such as current or gain level, to pump diode 805. The controller may be programmed in different ways, such as to apply a predetermined low current value to the pump diode to set oscillator operation in the single pulse regime, and other control functionality may be provided. Alternatively, an operator may observe the signal from the autocorrelator and set the gain or current of the pump diode such that the oscillator operates in the single-pulse regime.

In operation, the laser threshold current (i.e., the self-start current for mode locking) is initially established. The oscillator pump diode current is increased gradually from this mode-locking threshold and the photodiode signal displayed and/or analyzed for the presence of oscillator instability over a desired diode current range.

In the absence of detecting oscillator instability, the operating power of the oscillator can be set at a level of preference, e.g., about twice the start power measured at the mode-locking threshold.

An increase in gain in the oscillator cavity, such as by increasing the current of the pump diode, increases the overall output power of the laser. At some point, the gain is high enough that the oscillator could produce two or multiple pulses per round trip and remain mode locked. The temporal occurrence of the second or additional pulses resulting from multiple mode instability, relative to the first pulse, changes with the laser gain and may even show a random behavior.

High bi-stability transition occurs when an abrupt increase in photodiode signal amplitude takes place, or when an abrupt increase in auto-correlator pulse-width coupled with an abrupt decrease in its magnitude (measured, e.g., at zero delay time of the autocorrelator), appears with increasing diode current. Low transition is evaluated conversely in the presence of decreasing diode current. Stable laser oscillator performance can be achieved by setting the operating diode current at a level below the low limit of ascertained hi-stability, and, in an embodiment, above the level of the mode lock start current. One available current margin defined by the difference between the low transition and the self-start mode-locked currents is in excess of 75 mA. The output pulse-width and average power are different before and after each bistable transition.

Optical bi-stability has been observed in a number of non-UV, ultra-short pulse laser oscillators, and multiple bistable regions are also observed in a few cases. High transition incurs an abrupt increase (“quantum jump”) in the laser pulse width and output power. Low transition incurs an abrupt decrease (“quantum fall”) in the laser pulse width and output power. The instantaneous, and not gradual, changes of laser parameters (pulse-width and output power) at the bistable transitions are atypical hysteretic behaviors. Total energy is found not to be conserved in the process of transitions.

For pulse-splitting due to over saturation of a mode-locking saturable absorbing element to occur, a discrete change in average power is not expected because of total energy conservation. Above the self-start threshold, the laser gain of the primary mode increases with excitation. The oscillator maintains stable single-mode operation and passes over the low limit of bi-stability where another competing mode surpassing its higher threshold of excitation begins to appear. At the high limit of bi-stability, gain saturation of the primary mode can no longer support further narrowing of the ultra-short pulses due to dispersion and self-phase modulation effects along the transmission medium channel inside the mode-locked resonant cavity. Consequently the competing mode providing higher gain saturation takes over. The total energy switches to a higher level because of the higher gain achievable with the new dominant mode. Further excitation of this mode persists until its gain is saturated and another new competing mode prevails, and another increase in total energy is observed.

As excitation and laser gain decrease, the dominant resonant mode continues to operate in the bi-stability region without switching back to its predecessor. The laser operation is unstable, especially when excitation draws close to the low limit of bi-stability where its gain declines to a low level and the gain of the preceding mode becomes significant. At the low transition, mode switching occurs and the total energy falls back to the energy of the preceding mode.

Optical bi-stability in a mode-locked laser oscillator affects the output performance of pulse amplification in terms of changing pulse-width and peak power, which are critical to applications including tissue ablation. These abrupt changes at the high and low bi-stability transitions can be observed and measured when an external auto-correlator is employed at the output of a chirped pulse amplifier (CPA) or master oscillator power amplifier (MOPA) laser system. A pulse propagation time delay due to optical path and amplifier switching occurs before the detection of an oscillator pulse variation at the far end of the laser amplification system. In certain instances, detection of an amplified pulse may result in an inherent time delay. Continuously varying the excitation power or pump diode current for detection of bistable transitions could therefore introduce errors if measurements are made at a point or points away from the output of an unstable oscillator.

In certain instances, detection of an amplified pulse may result in a time delay generally associated with a finite response time of the detection electronics. If the excitation power or oscillator pump diode current for detection of bistable transitions is scanned too fast, it could introduce errors in the detection of transition points. In one aspect of operation, the system initially identifies neighborhoods of high and low transitions by scanning discretely over the pump diode current at a reasonable interval, typically a small number of mA, for a duration longer than the time between laser pulses defined by the laser system frequency. This procedure is repeated once or more by each time restricting a narrow scan range in the vicinity of the transitions and setting a progressively finer scan current interval (e.g., 1.0 mA, 0.1 mA, . . . ) until the desired measurement accuracy is achieved. A fast photodiode, the built-in photodiode of an auto-correlator, a single-shot auto-correlator, any nonlinear detector whose output signal is proportional to the square (or cube or higher power) of the peak power of the pulses (for example, based on second-or-higher-harmonic generation or based on two- or multiple-photon absorption), or an optical multi-channel spectrum analyzer can be used to capture the pulse variation in the presence of a bistable transition.

in other words, in one aspect, the present design scans oscillator gain and monitors auto-correlator response, such as in a case where the pump diode current is changing (increasing or decreasing). The auto-correlator may detect transitions from single to double and double to single pulse variations. In the case of a desired single pulse regime, pump diode current may be set at a value below the double pulse to single pulse transition to ensure single pulse operation (i.e., below the bistable regime as defined above). Conversely, in a situation where a double pulse is desired, the measurement of the nonlinearity may be employed and pump current may be set at a point above the single pulse to double pulse transition to ensure double pulse operation.

Thus, one embodiment of the present design provides a laser oscillator having an extracavity waveplate to spectrally filter pump laser diode radiation and an optional intracavity waveplate to limit competing resonant modes, both tilted and rotated to remove nonlinear anomalies encountered during mode transitions. In another embodiment, the present design comprises a laser engine including an oscillator and a photosensor, such as a fast photodetector or an auto-correlator, positioned to receive a beam of laser light associated with the oscillator or laser engine, and a controller configured to receive readings from the photosensor and alter laser gain provided within the oscillator to a level outside an unstable performance zone to avoid bistable oscillator operation.

In another embodiment, multiple components may be repositioned, and in another embodiment, certain components may be replaced with other components or removed form or inserted into the beam path using computer control. Mechanical stages may be employed with any components in a pulse stretcher/pulse compressor or elsewhere in the beam path including but not limited to gratings, prisms, grisms, reflective surfaces or mirrors, half wave plates, lens assemblies or focusing lenses, retro-reflect assemblies, Faraday isolators, folding mirrors, half mirrors, energy wheels, and/or dispersion elements.

An apparatus implementing the techniques or components described herein may be a stand-alone device or may be part of a larger device.

The previous description of the disclosure is provided to enable any person skilled in the art to make or use the disclosure. Various modifications to the disclosure will be readily apparent to those skilled in the art, and the generic principles defined herein may be applied to other variations without departing from the scope of the disclosure. Thus, the disclosure is not intended to be limited to the examples and designs described herein but is to be accorded the widest scope consistent with the principles and novel features disclosed herein.

What is claimed is:

1. A surgical system configured to deliver a pulsed laser beam to a patient's eye, comprising:
a laser engine, having
a laser oscillator comprising a pump diode configured to direct laser energy and a laser resonant cavity; and
a waveplate positioned external to the laser resonant cavity and in front of the pump diode, the waveplate tilted and rotated to remove nonlinear anomalies encountered during mode transitions of the oscillator.

2. The surgical system of claim 1, wherein the waveplate is a half-wave crystal quartz retardation plate.

3. The surgical system of claim 1, wherein the waveplate is a quarter-wave crystal quartz retardation plate.

4. The surgical system of claim 1, wherein the waveplate is an eighth-wave crystal quartz retardation plate.

5. The surgical laser system of claim 1, wherein the laser engine further includes a second waveplate positioned inside the laser resonant cavity, the second waveplate tilted and rotated to define a laser wavelength bandwidth for single mode operation of the laser oscillator.

6. The surgical laser system of claim 1, wherein the surgical laser system is a non-ultraviolet, ultra-short pulsed laser system.

7. A method for delivering a pulsed laser beam to a patient's eye using a laser engine having a laser oscillator and a waveplate, the method comprising:
generating a pulsed laser beam;
directing laser energy and a laser resonant cavity by the laser oscillator; and
removing nonlinear anomalies encountered during mode transitions of the oscillator by the waveplate.

8. The method of claim 7, wherein the laser oscillator comprises a pump diode.

9. The method of claim 8, wherein the waveplate is positioned external to the laser resonant cavity and in front of the pump diode.

10. The method of claim 7, wherein the waveplate is tilted and rotated.

11. The method of claim 7, wherein the waveplate is a half-wave crystal quartz retardation plate.

12. The method of claim 7, wherein the waveplate is a quarter-wave crystal quartz retardation plate.

13. The method of claim 7, wherein the waveplate is an eighth-wave crystal quartz retardation plate.

14. The method of claim 7, wherein the laser engine further includes a second waveplate positioned inside the laser resonant cavity, the second waveplate tilted and rotated to define a laser wavelength bandwidth for single mode operation of the laser oscillator.

15. The method of claim 7, wherein the laser engine is a non-ultraviolet, ultra-short pulsed laser engine.

16. A surgical system configured to deliver a pulsed laser beam to a patient's eye, comprising:
a laser engine, having:
an oscillator;
a photosensor configured to receive a laser beam associated with the oscillator, and
a controller configured to receive readings from the photosensor and to alter the laser gain provided within the oscillator to a level outside an unstable performance zone to avoid anomalous oscillator operation.

17. The surgical system of claim 16 further comprises an auto-correlator, a pulse stretcher/compressor, and an amplifier.

18. The surgical system of claim 16 further comprises a fast photodetector, a pulse stretcher/compressor, and an amplifier.

19. The surgical system of claim 17, wherein the controller is further configured to receive readings from the sensor and to alter an oscillator operating parameter to produce a single pulse per round-trip.

20. The surgical laser system of claim 16, wherein the surgical laser system is a non-ultraviolet, ultra-short pulsed laser system.
